(19) United States
(12) Patent Application Publication
(10) Pub. No.: US 2009/0264866 A1
(43) Pub. Date: Oct. 22, 2009

(54) Catheter Identification Inserts

(75) Inventor: August Powell, Zimmerman, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/105,858

(22) Filed: Apr. 18, 2008

(51) Int. Cl. A61M 25/18

(52) U.S. Cl. 604/533

(57) ABSTRACT

An intravascular catheter that may include an elongate shaft having a proximal end and a lumen extending distally from the proximal end, a hub connected to the shaft lumen, the hub including a body portion extending along a longitudinal axis from a proximal end of the hub to a distal end of the hub, the body portion including a lumen fluidly connected to the shaft, the hub further including a first wing portion and a second wing portion, each wing portion extending along the longitudinal axis, wherein the body portion is made from a monolithic transparent component and wherein each wing portion is partially made from the monolithic transparent component and partially made from an opaque component, the opaque component forming a first flat surface of the hub and being non-removable from the monolithic transparent component and visual or electronic information disposed on or in the hub.

### TECHNICAL FIELD

The present invention pertains to intravascular devices such as catheters and apparatuses and methods for visual and electronic identification thereof.

### BACKGROUND

The present invention relates to the field of catheterization of lumens within the human body, particularly lumens in the cerebral, peripheral and heart vasculature. The invention has application to guide, diagnostic, drug delivery, balloon and other therapeutic catheters.

Many medical procedures involve the insertion of one or more catheters into a lumen of a living body. These catheters usually include a luer hub and/or a manifold at the proximal end for grasping and for providing interfaces with other devices. These luer hubs and manifolds can be transparent and clear to be able to see any undesirable air bubbles. This clarity along with necessary size restrictions makes it difficult to provide all the desired information on the luer hub or manifold in a manner that can be easily and efficiently read. Thus is there an ongoing need for alternative catheter designs and manufacturing processes.

### SUMMARY

One embodiment pertains to a catheter having a transparent catheter hub having wings at least a portion of which are substantially opaque. Information such as product information can be legibly marked on the substantially opaque portion with laser-marking or other technique. A radio-frequency identification (RFID) microchip may be disposed in the substantially opaque portion of the wings or other suitable part of the catheter hub.

Another embodiment pertains to a method of making such a catheter in which a transparent hub body portion is provided where the body portion extends at least partially into the hub wings and where a substantially opaque material is overmolded to form the hub wings or to snap-fit to the hub wings.

The above summary of some embodiments is not intended to describe each disclosed embodiment or every implementation of the present invention. The Figures and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of the proximal portion of a representative catheter having a hub;

FIG. 2 is a perspective exploded view of the proximal portion of the catheter of FIG. 1;

FIG. 3 is a perspective exploded view of the proximal portion of a variation of the catheter of FIG. 1; and

FIG. 4 is a perspective view of a representative catheter identification insert.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

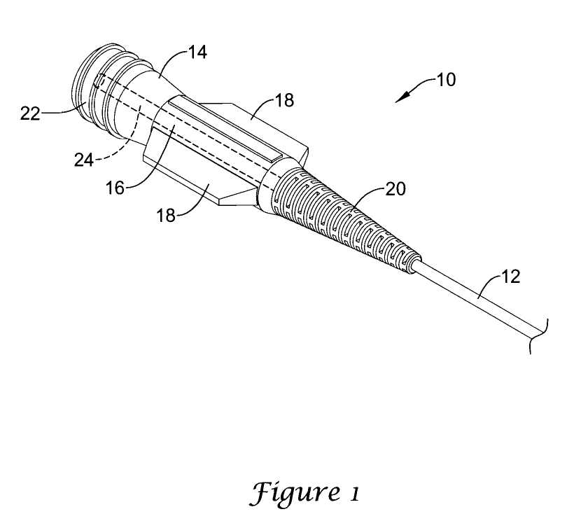

FIG. 1 is a schematic view of a catheter 10, which has a shaft 12 and a hub 14. Shaft 12 has a proximal end and a distal end and a lumen extending therethough. Hub 14 has a transparent hub body 16 that is fluidly connected to shaft 12 and hub wings 18 that are at least partially opaque. The catheter hub may also include other features such as a strain relief member 20 and a luer fitting 22.

Catheter 10 is a simple single lumen catheter with a simple hub design; it should not be thought, however, that the invention is so limited to this example embodiment. Embodiments of the invention may be included in a wide variety of catheters including multi-lumen catheters and catheters having a hub with multiple ports and one or more wings or catheters having manifolds or other hub-likes structures. Features not specifically discussed herein such as shaft construction, strain relief or luer port design or methods of use should not be considered limiting. For example, a contemplated embodiment of the invention is a balloon catheter where a hub has an inflation port and a guidewire port, each connected by a separate hub lumen to a separate shaft lumen.

Hub body 16 has a lumen 24 extending therethrough fluidly connected to the lumen of shaft 12 and is transparent along substantially the whole length of lumen 24 to allow for visual inspection. Wings 18 extend along the longitudinal axis of the hub to permit orientation and identification of the catheter.

Wings 18 include an opaque portion to permit catheter information to be legibly marked thereon. Such information (not depicted in the Figure) may be printed, laser-marked or marked using some other suitable technique and may include product name, pertinent characteristics such as length or outer diameter, product part number, serial number, manufacturing or use-by date, one- or two-dimensional bar codes or other desired information. Further, the opaque portion may be made from a polymer of a particular color where the color is selected to correspond to a pertinent characteristic of the catheter such as length, outer diameter or lumen diameter.

In addition or as an alternative to the visual display of information on the hub, an RFID (radio frequency identification) microchip may be disposed in the hub. Such a chip can hold much more information about the catheter than could be legibly printed on the surface of the hub and can be easily read by an electronic device and displayed. The electronic device, by reading the RFID microchip, puts the information into an electronic form that can be leveraged by computer and interconnectivity technologies. For example, current information about the product, such as devices currently compatible with the catheter, product recall or warning information, or detailed instructions for use can be displayed in a timely manner for the user. The RFID information can also be easily added to the electronic record of the procedure.

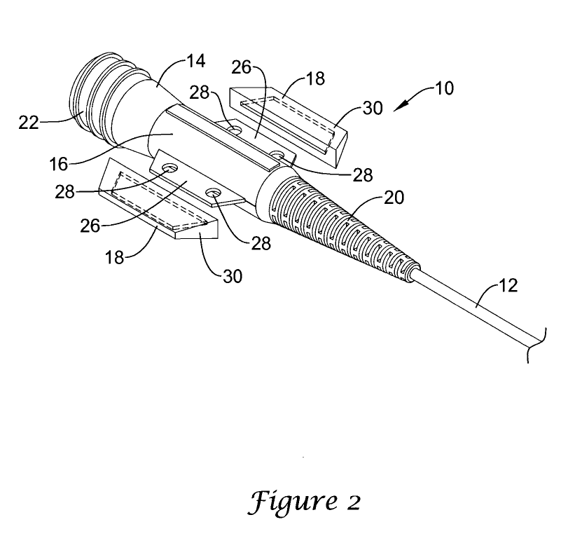

One example method of making such a catheter is illustrated by reference to FIG. 2, which is an exploded diagrammatic view of a catheter hub 14. Catheter hub 14 has a monolithic transparent body portion 16 that can be made through an injection molding or other suitable process. The body portion 16 can be formed over a catheter shaft or can be made separately from the catheter shaft and attached at a subsequent step. Body portion 16 extends at least partially into hub wings 18. In this embodiment, hub wings 18 include portion 26 of the body portion 16 and also include opaque portion 30. Portion 26 may include holes 28 or other geometry to enhance adhesion of the other portions of the wings. A tinted, opaque or colored polymer is selected for use. The polymer need not be completely opaque but should be selected with a view toward the legibility of the printed information. For example, a partially opaque material may work in some applications. The body portion 16 is placed in a mold and opaque portions 30 are molded onto the body portion. The opaque portion can be produced with a flat surface and can be printed or laser-marked with desired information.

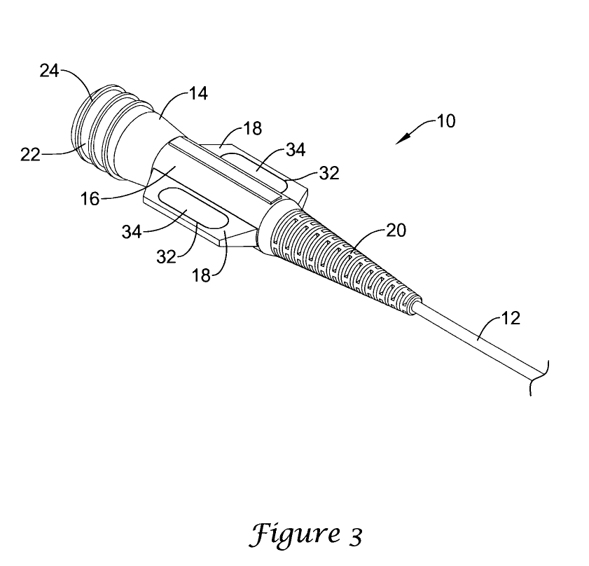

Another example method of making such a catheter is illustrated by reference to FIG. 3, which is a schematic view of a catheter hub 14. Catheter hub 14 has a monolithic transparent body portion 16 that includes first and second wings 18. In this embodiment, each wing 18 includes an opening 32 for the insertion of a product identification insert. In another contemplated embodiment, only one wing has an opening for a product identification insert. Separately produced product identification inserts 34 are configured to be snap-fit into the one or more openings 32. The inserts shown are substantially the same size and shape of the openings. However, that need not be the case. For example, an insert that has substantially the shape of the whole wing may be formed having a central peg to be snap fit into the opening on the wing. Such product identification inserts may be made by any suitable process such as injection molding or machining and may be marked with desired information prior or subsequent to installing it to the hub.

Other example methods of producing such a catheter include the following. An opaque identification insert may be affixed to a wing by adhesive or thermal bonding, heat staking, or other suitable technique. In another embodiment, two opaque portions may be joined by adhesion or a snap fit or the like over a body portion of a wing. In another embodiment, opaque wings may be produced separately from the catheter body and affixed by adhesive or thermal bonding methods.

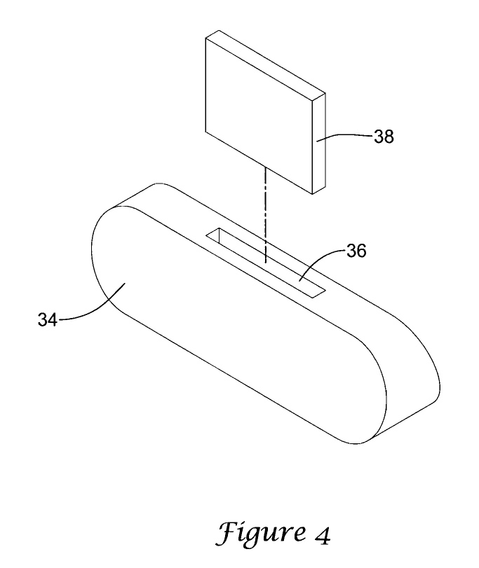

An RFID chip may be installed into any of the above catheter configurations by any suitable technique. In one technique, illustrated in FIG. 4, a cavity 36 is formed in identification insert 34 into which an RFID chip 38 is inserted prior to snap-fitting the insert into the opening 32 on the wing. A suitable cavity can also be formed in the hub body or between the hub body and the identification insert. Another example technique involves insert molding the RFID chip 38 into the identification insert prior to attaching the identification insert to the hub wing.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. An intravascular catheter, comprising an elongate shaft having a proximal end and a lumen extending distally from the proximal end; and
a hub connected to the shaft lumen, the hub including a body portion extending along a longitudinal axis from a proximal end of the hub to a distal end of the hub, the body portion including a lumen fluidly connected to the shaft, the hub further including a first wing portion and a second wing portion, each wing portion extending along the longitudinal axis of the hub, wherein the body portion is made from a monolithic transparent component and wherein each wing portion is partially made from the monolithic transparent component and partially made from an opaque component, the opaque component forming a first flat surface of the hub and being non-removable from the monolithic transparent component.

2. The catheter of claim 1, wherein the first wing portion is partially made from a first opaque component and wherein the second wing portion is partially made from a second opaque component.

3. The catheter of claim 1, wherein the first and second wing portions are on opposing sides of the hub.

4. The catheter of claim 1, further comprising indicia marked on the first flat surface.

5. The catheter of claim 4, wherein the indicia is laser-marked.

6. The catheter of claim 4, wherein the indicia is printed.

7. The catheter of claim 1, wherein the opaque component has a color selected to correspond with a feature of the catheter.

8. The catheter of claim 7, wherein the catheter has a length and wherein the feature is the catheter length.

9. The catheter of claim 7, wherein the shaft lumen has a diameter and wherein the feature is the shaft lumen diameter.

10. The catheter of claim 1, wherein the monolithic hub body includes a strain relief extending distally around the elongate shaft proximal end.

11. The catheter of claim 1, further comprising an RFID microchip embedded in the hub.

12. The catheter of claim 11, wherein the RFID microchip is disposed in the opaque component.

13. The catheter of claim 11, wherein the RFID microchip is disposed in a cavity between the opaque component and the monolithic transparent component.

14. The catheter of claim 1, wherein the opaque component is a product identification insert.

15. The catheter of claim 1, wherein the opaque component is snap-fitted onto the monolithic transparent component.

16. The catheter of claim 1, wherein the opaque component is overmolded onto the monolithic transparent component.

17. The catheter of claim 1, wherein the opaque component is adhesive bonded to the monolithic transparent component.

18. The catheter of claim 1, wherein the opaque component is heat staked to the monolithic transparent component.

19. The catheter of claim 1, wherein the opaque component is completely opaque.

20. The catheter of claim 1, wherein the opaque portion has at least 85 percent opacity.

21. A method of producing a catheter comprising the steps of:
providing a monolithic transparent hub body having a lumen extending therethrough and first and second wing portions; and
disposing an opaque product identification insert on the first and second wing portions, the product identification insert having catheter identification information.

22. The method of claim 21, further comprising the step of putting the catheter identification information on the product identification insert prior to the step of disposing the product identification insert on the first and second wing portions.

23. The method of claim 21, further comprising the step of putting the catheter identification information on the product identification insert subsequent to the step of disposing the product identification insert on the first and second wing portions.

24. The method of claim 21, wherein the product identification insert further comprises an RFID microchip.

25. The method of claim 21, wherein the product identification information includes a color of the product identification insert selected to correspond with a functional feature of the catheter.

26. The method of claim 21, wherein the step of disposing the product identification insert includes the step of snap-fitting the product identification insert to the monolithic transparent hub body.

27. The method of claim 21, wherein the step of disposing the product identification insert includes the step of overmolding the product identification insert on the monolithic transparent hub body.
